(19) United States
(12) Patent Application Publication
(10) Pub. No.: US 2004/0249281 A1
(43) Pub. Date: Dec. 9, 2004

(54) Method and apparatus for extracting wall function information relative to ultrasound-located landmarks

(76) Inventor: Bjorn Olstad, Stathelle (NO)

Correspondence Address: MCANDREWS HELD & MALLOY, LTD, 500 WEST MADISON STREET, SUITE 3400, CHICAGO, IL, 60661

(21) Appl. No.: 10/848,721

(22) Filed: May 19, 2004

Related U.S. Application Data
(60) Provisional application No. 60/477,182, filed on Jun. 9, 2003.

(51) Int. Cl. A61B 8/00

(52) U.S. Cl. 600/437; 600/450

(57) ABSTRACT

An ultrasound machine is disclosed that includes a method and apparatus for generating an image responsive to moving cardiac structure, and for extracting wall function information relative to anatomical landmarks located within the heart. At least one processor is responsive to signals received from the heart to locate anatomical landmarks within the cardiac structure and generate position information of the anatomical landmarks, locate walls within the heart relative to the position information of the anatomical landmarks, and extract wall function information from the walls within the heart. The landmarks, walls, and wall function information may be displayed to a user of the ultrasound machine.

### RELATED APPLICATIONS/INCORPORATION BY REFERENCE

This application is related to, and claims benefit of and priority from, Provisional Application No. 60/477,182 dated Jun. 9, 2003 (Attorney Docket No. 15-DS-00551 (131111US01) titled “Extracting Wall Function Information Relative to Ultrasound-Located Landmarks”, the complete subject matter of which is incorporated herein by reference in its entirety.

The complete subject matter of each of the following U.S. Patent Applications is incorporated by reference herein in their entirety:

U.S. patent application Ser. No. 10/248,090 filed on Dec. 17, 2002.

U.S. Patent Application Ser. No. 10/064,032 filed on Jun. 4, 2002.

U.S. patent application Ser. No. 10/064,083 filed on Jun. 10, 2002.

U.S. patent application Ser. No. 10/064,033 filed on Jun. 4, 2002.

U.S. patent application Ser. No. 10/064,084 filed on Jun. 10, 2002.

U.S. patent application Ser. No. 10/064,085 filed on Jun. 10, 2002.

### FEDERALLY SPONSORED RESEARCH OR DEVELOPMENT

[Not Applicable]

### BACKGROUND OF THE INVENTION

Echocardiography is a branch of the ultrasound field that is currently a mixture of subjective image assessment and extraction of key quantitative parameters. Evaluation of cardiac wall function has been hampered by a lack of well-established parameters that may be used to increase the accuracy and objectivity in the assessment of, for example, coronary artery diseases. Stress echo is such an example. It has been shown that the subjective part of wall motion scoring in stress echo is highly dependent on operator training and experience. It has also been shown that inter-observer variability between echo-centers is unacceptably high due to the subjective nature of the wall motion assessment.

Much technical and clinical research has focused on the problem and has aimed at defining and validating quantitative parameters. Encouraging clinical validation studies have been reported, which indicate a set of new potential parameters that may be used to increase objectivity and accuracy in the diagnosis of, for instance, coronary artery diseases. Many of the new parameters have been difficult or impossible to assess directly by visual inspection of the ultrasound images generated in real-time. The quantification has typically required a post-processing step with tedious, manual analysis to extract the necessary parameters. Determination of the location of anatomical landmarks in the heart is no exception. Time intensive post-processing techniques or complex, computation-intensive real-time techniques are undesirable.

A method in U.S. Pat. No. 5,601,084 to Sheehan et al. describes imaging and three-dimensionally modeling portions of the heart using imaging data. A method in U.S. Pat. No. 6,099,471 to Torp et al. describes calculating and displaying strain velocity in real time. A method in U.S. Pat. No. 5,515,856 to Olstad et al. describes generating anatomical M-mode displays for investigations of living biological structures, such as heart function, during movement of the structure. A method in U.S. Pat. No. 6,019,724 to Gronningsaeter et al. describes generating quasi-realtime feedback for the purpose of guiding procedures by means of ultrasound imaging.

### BRIEF SUMMARY OF THE INVENTION

An embodiment of the present invention provides an ultrasound system for imaging a heart and extracting wall function information from the heart after having automatically located anatomical landmarks within the heart.

At least one embodiment of the present invention enables real-time extraction of wall function information within a heart, including temporal variations in wall motion and wall thickening, after locating and tracking certain anatomical landmarks of the heart. Moving cardiac structure is monitored to accomplish the function. An embodiment of the present invention helps establish improved, real-time visualization and assessment of wall function parameters of the heart. The moving structure is characterized by a set of analytic parameter values corresponding to anatomical points within a myocardial segment of the heart. The set of analytic parameter values may comprise, for example, tissue velocity values, time-integrated tissue velocity values, B-mode tissue intensity values, tissue strain rate values, blood flow values, and mitral valve inferred values.

One embodiment of the present invention comprises a method for generating an image responsive to moving structure. This embodiment comprises locating at least one landmark within the structure and generating position information of at least the one landmark. The method further comprises locating at least one wall relative to the position information of the at least one landmark extracts function information from the at least one wall. It is further contemplated that the method may comprise displaying at least the function information.

An apparatus is provided in an ultrasound machine for imaging a heart and extracting wall function information from the heart relative to certain anatomical landmarks within the heart. In such an environment an apparatus adapted to extract the wall function information comprises a front-end arranged to transmit ultrasound waves into a structure and to generate received signals in response to ultrasound waves backscattered from the structure over a time period.

It is contemplated that the apparatus may comprise a processor responsive to the received signals to generate a set of analytic parameter values representing movement of the cardiac structure over the time period and analyzes elements of the set of analytic parameter values to automatically extract position information of the anatomical landmarks and track the positions of the landmarks.

It is further contemplated that the apparatus may comprise a processor responsive to the tracked anatomical landmark positions and extracts wall function information from certain locations within the heart relative to the tracked anatomical landmarks. A display is arranged to overlay indicia corresponding to the position information onto an image of the moving structure to indicate to an operator the position of the tracked anatomical landmarks, and to display the extracted wall function information.

In at least one embodiment, the apparatus comprises a display processor and monitor to process the position information and display indicia overlaying at least one of the at least one anatomical landmark, the at least one wall and/or the wall function information.

In at least one embodiment of the apparatus, the at least one anatomical landmark comprises at least one of an apex of the heart and an AV-plane of the heart. It is further contemplated that, in at least one embodiment, the at least one wall comprises at least one of a wall of a basal segment of the heart, a wall of a mid segment of the heart, a wall of at least one complete myocardial segment of the heart, a wall of at least one chamber of the heart, and a wall forming at least one boundary between at least two chambers of the heart. In at least one embodiment of the apparatus it is contemplated that the wall function information comprises at least one of peak systolic velocity, time to peak systolic velocity, velocity time integral for systole, peak E-velocity, peak A-velocity, E/A ratio, sound, and longitudinal motion.

It is further contemplated that, in at least one embodiment, the apparatus comprises at least one processor which comprises at least one of a Doppler processor, a non-Doppler processor, a control processor, and a PC back-end. It is further contemplated that the apparatus further comprises at least one transducer connected to the front-end adapted to convert electrical signals to the ultrasound waves and vice versa. In at least one embodiment, the apparatus further comprises at least one user interface connecting to the at least one processor to control operation of the ultrasound machine.

A method is also provided in an ultrasound machine for imaging a heart and extracting wall function information from the heart based on having previously located certain anatomical landmarks within the heart. In such an environment a method for extracting the wall function information comprises transmitting ultrasound waves into a structure and generating received signals in response to ultrasound waves backscattered from said structure over a time period. A set of analytic parameter values is generated in response to the received signals representing movement of the cardiac structure over the time period.

In at least one embodiment, position information of the anatomical landmarks is automatically extracted and the positions of the landmarks are then tracked. Wall function information is extracted from certain locations within the heart relative to the tracked anatomical landmarks. Indicia corresponding to the position information are overlaid onto the image of the moving structure to indicate to an operator the position of the tracked anatomical landmarks, and the extracted wall function information is also displayed.

One or more embodiments of the present invention comprise displaying the wall function information and/or displaying indicia overlaying the at least one anatomical landmark on a display of an ultrasound machine. In at least embodiment of the method of imaging a heart and extracting wall function information from the heart, the at least one anatomical landmark may comprise at least one of an apex of the heart and an AV-plane of the heart.

In at least one embodiment, the at least one wall comprises at least one wall of a basal segment of the heart, at least one wall of a mid segment of the heart, at least one wall of a myocardial segment of the heart, at least one wall of a chamber of the heart, and at least one wall forming a boundary between at least two chambers of the heart. It is further contemplated that the wall function information comprises at least one of peak systolic velocity, time to peak systolic velocity, velocity time integral for systole, peak E-velocity, peak A-velocity, E/A ratio, sound, and longitudinal motion.

Other embodiments of the method wherein extracting comprises setting the position of at least one of a region-of-interest (ROI), a Doppler sample volume, and a M-mode with respect to said at least one wall within the heart. In at least one embodiment, tracking comprises at least one anatomical landmark in position while locating at least one wall and the extracting wall function information. It is further contemplated comprising tracking the at least one wall in position while performing the extracting wall function information.

Certain embodiments of the present invention afford an approach to extract wall function information from a heart after automatically locating key anatomical landmarks of the heart, such as the apex and the AV-plane.

### BRIEF DESCRIPTION OF THE DRAWINGS

FIG. 1 is a diagram of an embodiment of an ultrasound machine made in accordance with various aspects of the present invention.

FIG. 2 is a flowchart of an embodiment of a method performed by the machine shown in FIG. 1, in accordance with various aspects of the present invention.

FIG. 3 is a diagram illustrating using the method of FIG. 2 in the ultrasound machine of FIG. 1 to identify wall tissue within a heart and to extract wall function information, in accordance with an embodiment of the present invention.

FIG. 4 is a diagram illustrating using the method of FIG. 2 in the ultrasound machine of FIG. 1 to extract sound information from a wall within a heart, in accordance with an embodiment of the present invention.

FIG. 5 is a diagram illustrating a non-apical view of a heart and landmark identification in non-apical views, in accordance with an embodiment of the present invention.

FIG. 6 is a diagram illustrating normal peak values at peak exercise in dobutamine stress in an apical 4-chamber view of a heart.

FIG. 7 shows the same information as FIG. 6 for the apical 2-chamber view of the heart.

FIG. 8 illustrates response curves for peak systolic velocities as a function of dobutamin level in a normal population.

FIG. 9 is a diagram of a heart illustrating how basal velocities are higher than, for example, velocities measured in the mid ventricle.

The foregoing summary, as well as the following detailed description of certain embodiments of the present invention, will be better understood when read in conjunction with the appended drawings. It should be understood, however, that the present invention is not limited to the arrangements and instrumentality shown in the attached drawings.

### DETAILED DESCRIPTION OF THE INVENTION

An embodiment of the present invention enables the real-time extraction of wall function information within a heart, including temporal variations in wall motion and wall thickening, after locating and tracking certain anatomical landmarks of the heart. Moving cardiac structure is monitored to accomplish the function. As used in the specification and claims, structure means non-liquid and non-gas matter, such as cardiac wall tissue. An embodiment of the present invention helps establish improved, real-time visualization and assessment of wall function parameters of the heart. The moving structure is characterized by a set of analytic parameter values corresponding to anatomical points within a myocardial segment of the heart. The set of analytic parameter values may comprise, for example, tissue velocity values, time-integrated tissue velocity values, B-mode tissue intensity values, tissue strain rate values, blood flow values, and mitral valve inferred values.

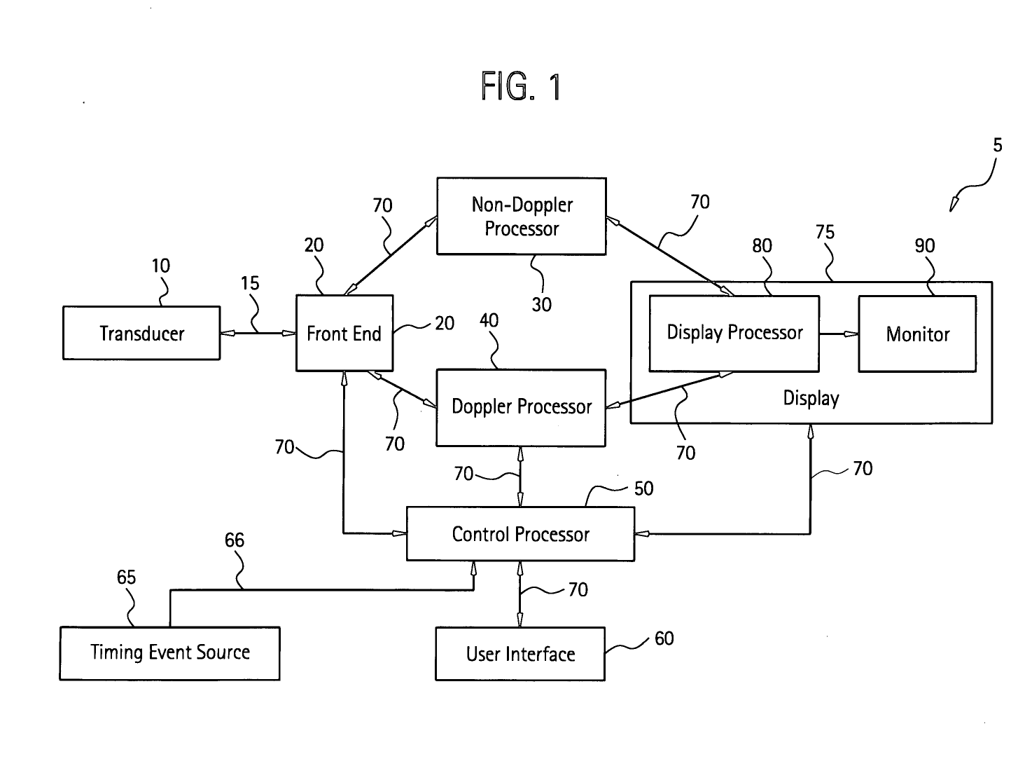

FIG. 1 is a diagram of an embodiment of the present invention comprising an ultrasound machine 5. A transducer 10 is used to transmit ultrasound waves into a subject by converting electrical analog signals to ultrasonic energy and to receive ultrasound waves backscattered from the subject by converting ultrasonic energy to analog electrical signals. A front-end 20 comprising a receiver, transmitter, and beamformer, is used to create the necessary transmitted waveforms, beam patterns, receiver filtering techniques, and demodulation schemes that are used for the various imaging modes. Front-end 20 performs the functions by converting digital data to analog data and vice versa. Front-end 20 interfaces at an analog interface 15 to transducer 10 and interfaces over a digital bus 70 to a non-Doppler processor 30 and a Doppler processor 40 and a control processor 50. Digital bus 70 may comprise several digital sub-buses, each sub-bus having its own unique configuration and providing digital data interfaces to various parts of the ultrasound machine 5.

Non-Doppler processor 30 comprises amplitude detection functions and data compression functions used for imaging modes such as B-mode, B M-mode, and harmonic imaging. Doppler processor 40 comprises clutter filtering functions and movement parameter estimation functions used for imaging modes such as tissue velocity imaging (TVI), strain rate imaging (SRI), and color M-mode. The two processors, 30 and 40, accept digital signal data from the front-end 20, process the digital signal data into estimated parameter values, and pass the estimated parameter values to processor 50 and a display 75 over digital bus 70. The estimated parameter values may be created using the received signals in frequency bands centered at the fundamental, harmonics, or sub-harmonics of the transmitted signals in a manner known to those skilled in the art.

Display 75 comprises scan-conversion functions, color mapping functions, and tissue/flow arbitration functions, performed by a display processor 80 which accepts digital parameter values from processors 30, 40, and 50, processes, maps, and formats the digital data for display, converts the digital display data to analog display signals, and passes the analog display signals to a monitor 90. Monitor 90 accepts the analog display signals from display processor 80 and displays the resultant image to the operator on monitor 90.

A user interface 60 allows user commands to be input by the operator to the ultrasound machine 5 through control processor 50. User interface 60 comprises a keyboard, mouse, switches, knobs, buttons, track ball, and on screen menus.

A timing event source 65 is used to generate a cardiac timing event signal 66 that represents the cardiac waveform of the subject. The timing event signal 66 is input to ultrasound machine 5 through control processor 50.

Control processor 50 is the main, central processor of the ultrasound machine 5 and interfaces to various other parts of the ultrasound machine 5 through digital bus 70. Control processor 50 executes the various data algorithms and functions for the various imaging and diagnostic modes. Digital data and commands may be transmitted and received between control processor 50 and other various parts of the ultrasound machine 5. As an alternative, the functions performed by control processor 50 may be performed by multiple processors, or may be integrated into processors 30, 40, or 80, or any combination thereof. As a further alternative, the functions of processors 30, 40, 50, and 80 may be integrated into a single PC backend.

Once certain anatomical landmarks of the heart are identified, (e.g., the AV-planes and apex as described in U.S. patent application Ser. No. 10/248,090 filed on Dec. 17, 2002) wall function information, such as temporal variations in wall motion and wall thickening, may be extracted and displayed to a user of the ultrasound system 5 in accordance with various aspects of the present invention. The various processors of the ultrasound machine 5 described above may be used to extract and display wall function information from various locations within the heart.

One embodiment of the present invention comprises a method for generating an image responsive to moving structure. This embodiment comprises locating at least one landmark within the structure and generating position information of at least the one landmark. The method further comprises locating at least one wall relative to the position information of the at least one landmark extracts function information from the at least one wall. It is further contemplated that the method may comprising displaying at least the function information.

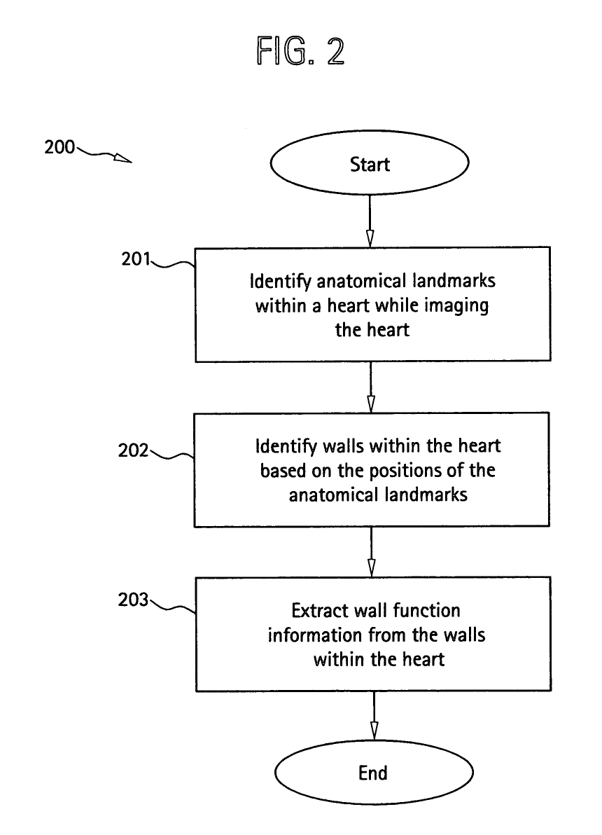

FIG. 2 is a flow chart of an embodiment of a method 200 performed by the machine 5 of FIG. 1 in accordance with various aspects of the present invention. In step 201, anatomical landmarks (e.g., the AV-plane and apex) are identified within the heart while imaging the heart. In step 202, walls of the heart are identified relative to the positions of the anatomical landmarks. In step 203, wall function information is extracted from the walls within the heart.

As defined herein, wall function information includes at least one of peak systolic velocity, time to peak systolic velocity, velocity time integral for systole, peak E-velocity, peak A-velocity, E/A ratio, sound, and longitudinal motion.

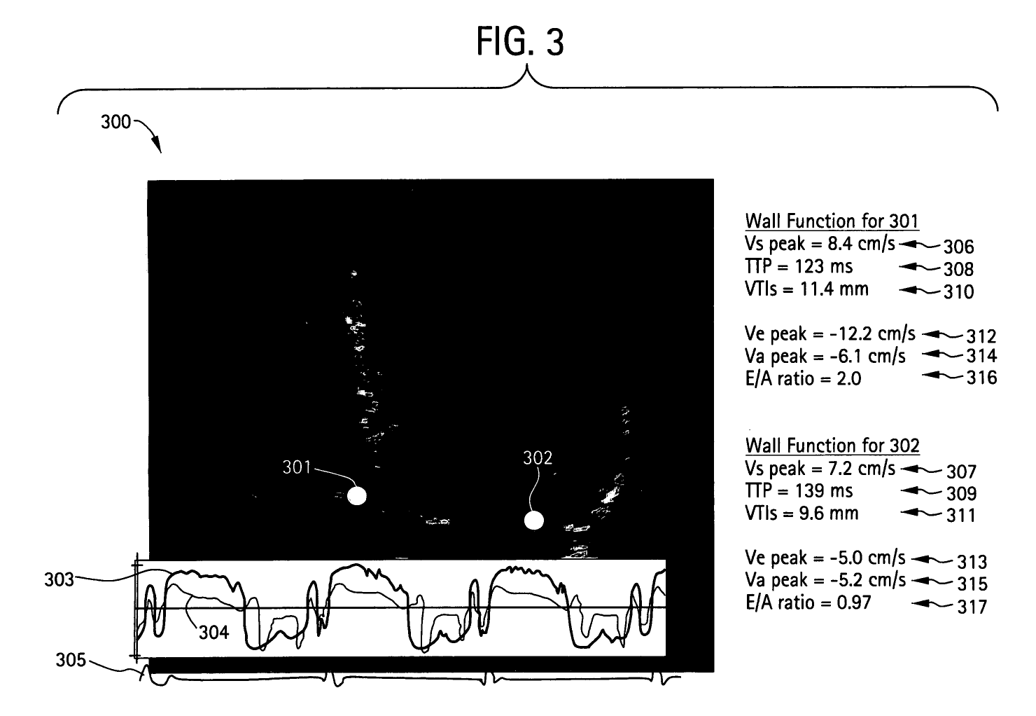

FIG. 3 is a diagram illustrating using the method 200 of FIG. 2 in the ultrasound machine 5 of FIG. 1 to identify wall tissue within a heart 300 and to extract wall function information, in accordance with an embodiment of the present invention. Detected landmarks may be used to identify walls within the heart given by relative positioning and local image characteristics. FIG. 3 illustrates a real-time application where a normal B-mode acquisition is conducted and, hidden for a user, tissue velocity information is gathered in a region of interest around the assumed AV-plane location. The two AV-plane locations are then identified in real-time and indicated with tracking markers 301 and 302. The associated velocity or strain rate profile may, therefore, be shown in real-time. The velocity profiles 303 and 304 are shown at the bottom of FIG. 3 synchronized with ECG 305. The extracted profiles 303 and 304 may also be analyzed in real-time with measurement algorithms to extract selected wall function parameters for one or both locations. The wall function parameters may include any aspect from the profiles. FIG. 3 illustrates wall function parameters such as peak systolic velocity 306 and 307, time to peak systolic velocity 308 and 309, velocity time integral for systole 310 and 311, peak E-velocity 312 and 313, peak A-velocity 314 and 315, and E/A ratio 316 and 317. In addition, longitudinal motion may indirectly be used to obtain a rough estimate of global ejection fraction.

In accordance with an embodiment of the present invention, the position of a region-of-interest (ROI), a Doppler sample volume, and a M-mode may be set with respect to a wall of the heart to extract wall function information. Also, anatomical landmarks and wall positions may be tracked to help with the extraction of the wall function information, in accordance with various embodiments of the present invention.

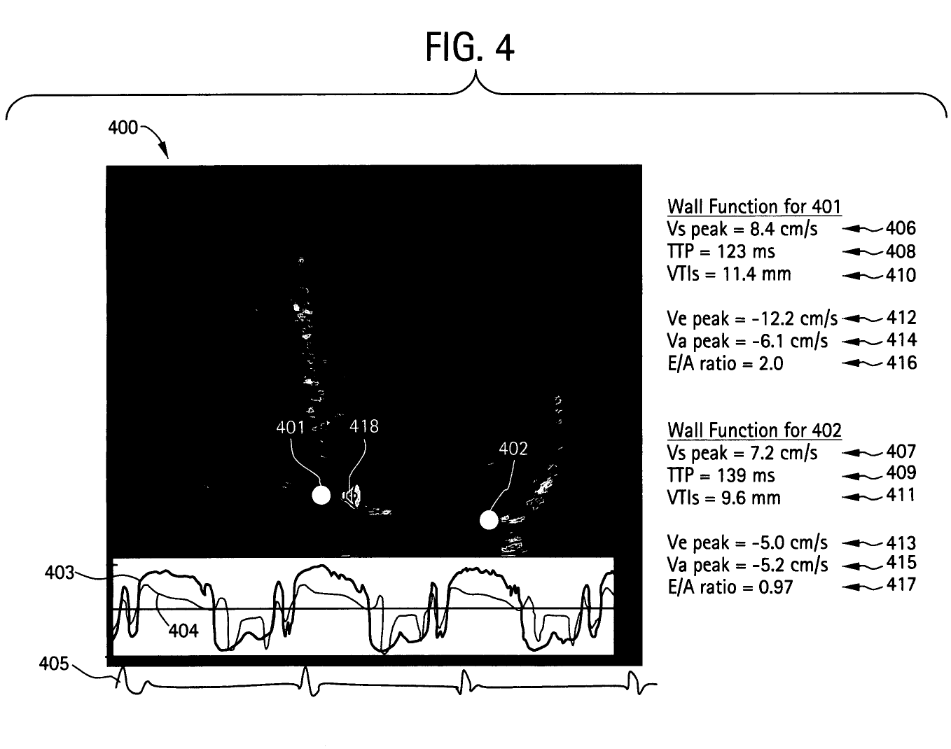

FIG. 4 is a diagram illustrating using the method 200 of FIG. 2 in the ultrasound machine 5 of FIG. 1 to extract sound information from a wall within a heart 400, in accordance with an embodiment of the present invention. FIG. 4 is identical to FIG. 3 but, in addition, the sound 418 for one selected AV-plane location 401 is extracted and may be used as a default sound during standard B-mode imaging. The two AV-plane locations are identified in real-time and indicated with tracking markers 401 and 402. The associated velocity or strain rate profile may, therefore, be shown in real-time. The velocity profiles 403 and 404 are shown at the bottom of FIG. 4 synchronized with ECG 405. The extracted profiles 403 and 404 may also be analyzed in real-time with measurement algorithms to extract selected wall function parameters for one or both locations. The wall function parameters may include any aspect from the profiles. FIG. 4 illustrates wall function parameters such as peak systolic velocity 406 and 407, time to peak systolic velocity 408 and 409, velocity time integral for systole 410 and 411, peak E-velocity 412 and 413, peak A-velocity 414 and 415, and E/A ratio 416 and 417.

The sound 418 is based on either variations in wall velocity or wall thickening. In accordance with an embodiment of the present invention, the frequency is shifted from the low frequencies corresponding to myocardial velocities to a range of frequencies that are more suitable for the human ear. Positive and negative velocities or strain rates may be separated into right and left audio channels of the ultrasound machine. For non-apical views, the associated location for audio extraction may be based on either maximal velocities or maximal strain rates. As a result, a user may hear the motion or contraction of the most active location in any view.

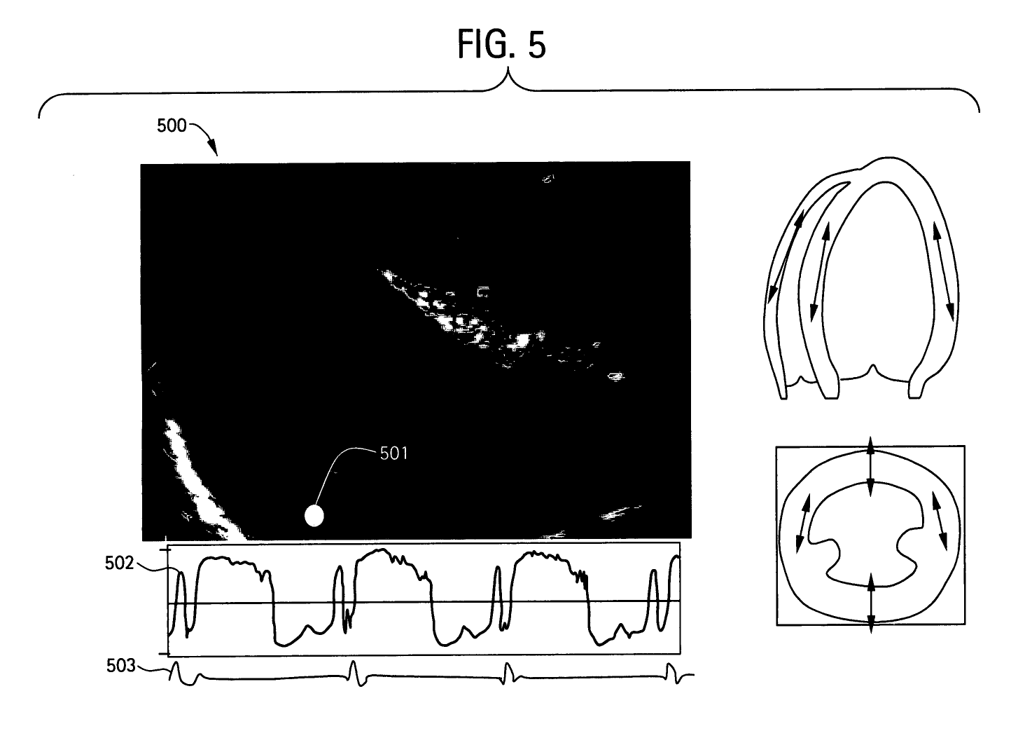

FIG. 5 is a diagram illustrating a non-apical view of a heart 500 and landmark identification in non-apical views, in accordance with an embodiment of the present invention. FIG. 5 shows an anatomical landmark 501 and an associated velocity profile 502 synchronized to ECG 503. Selection of peak velocity locations in terms of velocity of contraction may, for example, in a short-axis view, extract the upper and lower parts where the ultrasound beam is orthogonal to the wall contraction process.

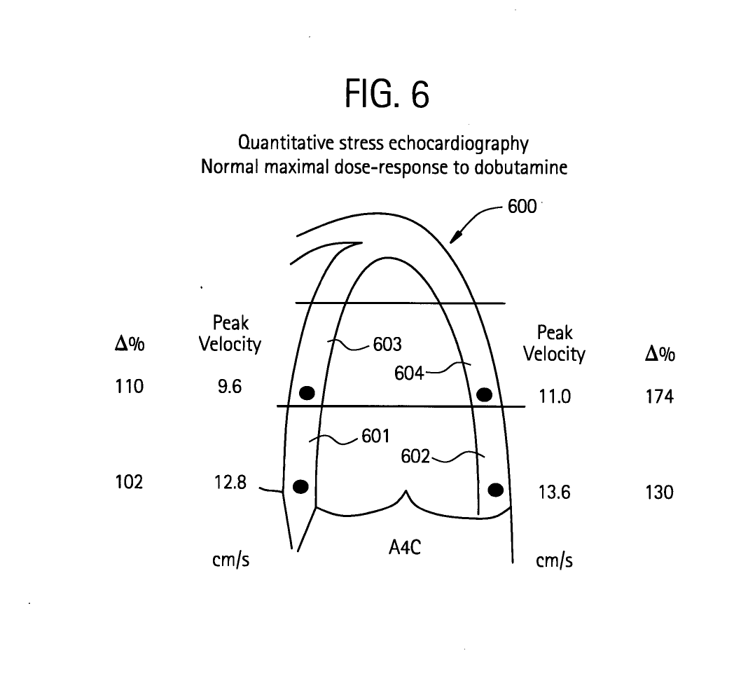

FIGS. 6, 7, and 8 illustrate academic work that has been performed for validation of peak systolic velocities as an indicator of, for example, ischemia in stress echo. The figures are taken from a European multi-center study headed by Dr. George Sutherland in Leuven, Belgium and Dr. Alan Fraser in Cardiff, Wales.

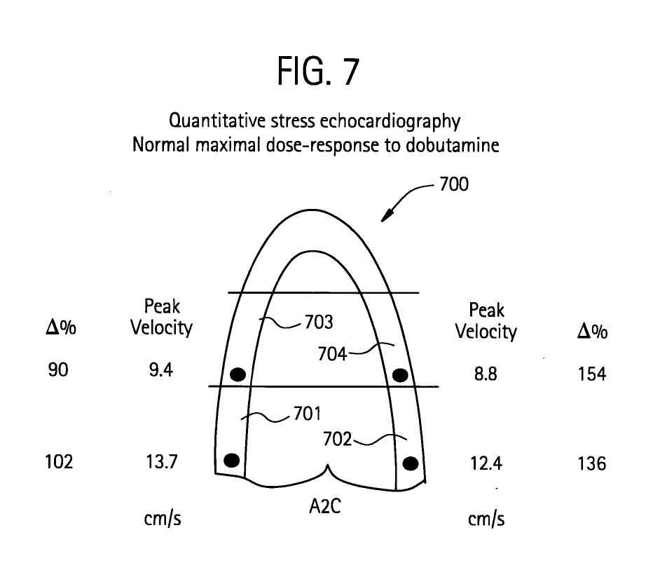

FIG. 6 is a diagram illustrating normal peak values at peak exercise in dobutamine stress in an apical 4-chamber view of a heart 600. The basal segments 601 and 602 are at about 13 cm/second and the mid segments 603 and 604 are at about 10-11 cm/second. The associated percentage changes indicate the relative change in peak systolic velocity relative to corresponding rest values. FIG. 7 shows the same information as FIG. 6 for the apical 2-chamber view of the heart 700. The basal segments 701 and 702 are at about 9 cm/second and the mid segments 703 and 704 are at about 12-14 cm/second.

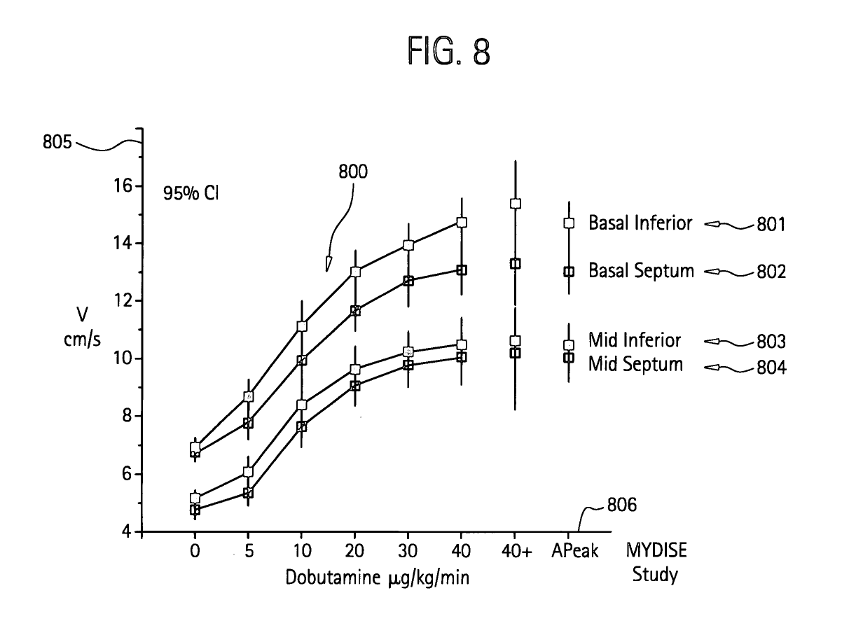

FIG. 8 illustrates the response curves 800 for peak systolic velocities 805 as a function of dobutamin level 806 in a normal population. FIG. 8 includes four locations that have been measured from apex. The four locations are basal inferior 801, basal septum 802, mid inferior 803, and mid septum 804. All the locations are able to approximately double the peak systolic velocity at peak exercise. Clinical results indicate that a reduction in peak systolic velocities at peak exercise is a good predictor of coronary artery diseases. The normal values indicated in FIGS. 6, 7, and 8 (or similar normal ranges) may be combined with the display techniques shown in FIGS. 3 and 4 to indicate normal values both textually together with the measured parameters, or graphically as a normal scaling factor for the velocity or strain rate profiles.

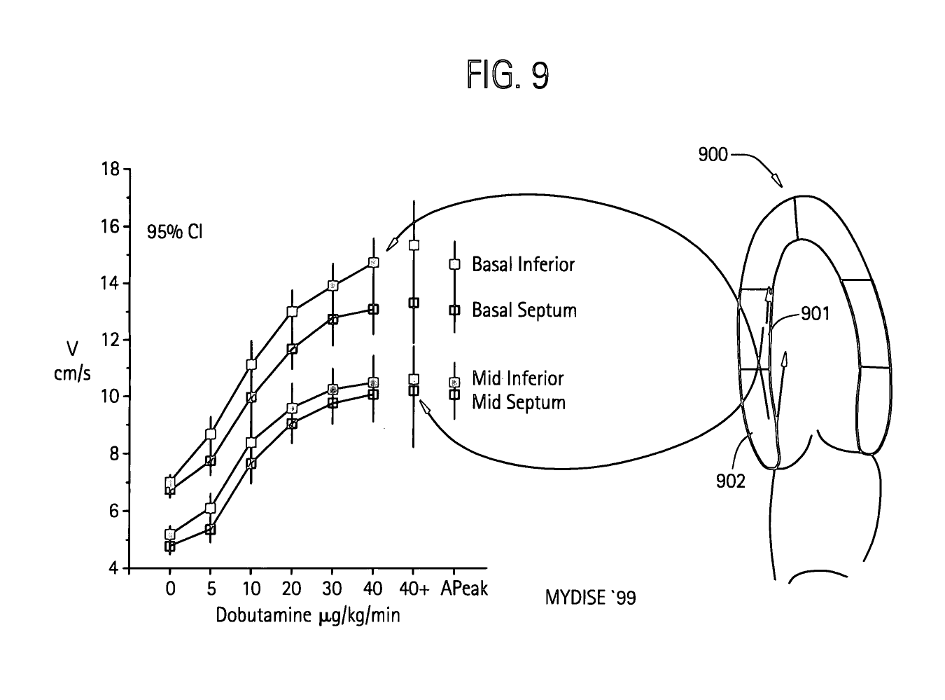

FIG. 9 is a diagram of a heart 900 illustrating how basal velocities are higher than, for example, velocities measured in the mid ventricle 901. Such a difference is caused by the contraction of the basal segment 902. The average strain rate or strain value for at least basal and mid segments may, therefore, be measured by measuring velocity variations at the upper and lower end of the segment. The landmark identification and associated geometrical locations may, therefore, be used to also provide real-time or post-processing assessments of regional strain.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for generating an image responsive to moving structure, a method comprising:
locating at least one landmark within the structure and generating position information of said at least one landmark;
locating at least one wall relative to said position information of said at least one landmark; and
extracting function information from said at least one wall.

2. The method of claim 1 comprising displaying at least said function information.

3. In an ultrasound machine for generating an image responsive to moving cardiac structure within a heart of a subject, a method comprising:
locating at least one anatomical landmark within the cardiac structure and generating position information of said at least one anatomical landmark;
locating at least one wall within the heart relative to said position information of said at least one anatomical landmark; and
extracting wall function information from said at least one wall within the heart.

4. The method of claim 3 further comprising displaying said wall function information on a display of said ultrasound machine.

5. The method of claim 3 further comprising displaying indicia overlaying said at least one anatomical landmark on a display of said ultrasound machine.

6. The method of claim 3 wherein said at least one anatomical landmark comprises at least one of an apex of the heart and an AV-plane of the heart.

7. The method of claim 3 wherein said at least one wall comprises at least one wall of a basal segment of the heart, at least one wall of a mid segment of the heart, at least one wall of a myocardial segment of the heart, at least one wall of a chamber of the heart, and at least one wall forming a boundary between at least two chambers of the heart.

8. The method of claim 3 wherein said wall function information comprises at least one of peak systolic velocity, time to peak systolic velocity, velocity time integral for systole, peak E-velocity, peak A-velocity, E/A ratio, sound, and longitudinal motion.

9. The method of claim 3 wherein said extracting comprises setting the position of at least one of a region-of-interest (ROI), a Doppler sample volume, and a M-mode with respect to said at least one wall within the heart.

10. The method of claim 3 further comprising tracking said at least one anatomical landmark in position while performing said locating at least one wall and said extracting wall function information.

11. The method of claim 3 further comprising tracking said at least one wall in position while performing said extracting wall function information.

12. In an ultrasound machine for generating an image responsive to moving cardiac structure within a heart of a subject, an apparatus comprising:
a front-end arranged to transmit ultrasound waves into the moving cardiac structure and blood and to generate received signals in response to ultrasound waves backscattered from the moving cardiac structure and blood;
at least one processor responsive to said received signals to locate at least one anatomical landmark within the cardiac structure and generate position information of said at least one anatomical landmark, locate at least one wall within the heart based on said position information of said at least one anatomical landmark, and extract wall function information from said at least one wall within the heart.

13. The apparatus of claim 12 further comprising a display processor and monitor to process said position information and display indicia overlaying at least one of said at least one anatomical landmark and said at least one wall.

14. The apparatus of claim 12 further comprising a display processor and monitor to process and display said wall function information.

15. The apparatus of claim 12 wherein said at least one anatomical landmark comprises at least one of an apex of the heart and an AV-plane of the heart.

16. The apparatus of claim 12 wherein said at least one wall comprises at least one of a wall of a basal segment of the heart, a wall of a mid segment of the heart, a wall of at least one complete myocardial segment of the heart, a wall of at least one chamber of the heart, and a wall forming at least one boundary between at least two chambers of the heart.

17. The apparatus of claim 12 wherein said wall function information comprises at least one of peak systolic velocity, time to peak systolic velocity, velocity time integral for systole, peak E-velocity, peak A-velocity, E/A ratio, sound, and longitudinal motion.

18. The apparatus of claim 12 wherein said at least one processor comprises at least one of a Doppler processor, a non-Doppler processor, a control processor, and a PC back-end.

19. The apparatus of claim 12 further comprising at least one transducer connected to said front-end to convert electrical signals to said ultrasound waves and vice versa.

20. The apparatus of claim 12 further comprising at least one user interface connecting to said at least one processor to control operation of said ultrasound machine.
